(19) United States
(12) Patent Application Publication
(10) Pub. No.: US 2026/0363647 A1
(43) Pub. Date: Nov. 19, 2026

(54) DETERMINING VALIDITY OF HEART SOUNDS SIGNALS FROM IMPLANTABLE MEDICAL DEVICE

(71) Applicant: Medtronic, Inc., Minneapolis, MN (US)

(72) Inventor: Subham Ghosh, Blaine, MN (US)

(21) Appl. No.: 19/135,708

(22) PCT Filed: Nov. 21, 2023
(86) PCT No.: PCT/IB2023/061728

§ 371 Date: Jun. 4, 2025

Related U.S. Application Data
(60) Provisional application No. 63/387,353, filed on Dec. 14, 2022.

(51) Int. Cl. A61B 7/02; A61B 5/00; A61B 7/04; A61N 1/365

(52) U.S. Cl. CPC A61B 7/023; A61B 5/7282; A61B 5/7285; A61B 7/04; A61N 1/36578

(57) ABSTRACT

A device, a system, and a process for determining the validity of a recorded episode of heart sounds are disclosed. A medical system may include a heart sounds sensor, an electrical sensor/stimulus for sensing or providing a ventricular electrical event, and a processor. The processor records an episode including a plurality of heart sounds segments, each heart sounds segment being triggered by an instance of the ventricular electrical event. For each heart sounds segment, the processor determines a correlation between the heart sounds segment and each other heart sounds segment in the episode. A heart sounds segment is considered a valid sample if the median correlation value with each of the other heart sounds segments in the episode is greater than a threshold. The episode is considered to have meaningful physiological data if greater than a threshold number of the heart sounds segments are characterized as valid samples.

This application claims the benefit of U.S. Provisional Patent Application Ser. No. 63/387,353, filed Dec. 14, 2022, the entire content of which is incorporated herein by reference.

### TECHNICAL FIELD

This disclosure generally relates to medical devices and, more particularly, to devices configured to sense a heart sounds signal.

### BACKGROUND

Heart sounds provide important diagnostic information relating to heart failure. For example, acoustic sensing of heart sounds using a stethoscope is a time-tested technique for a clinician to detect cardiac output and diagnose heart problems. Some examples of implantable medical devices (IMDs) include implantable cardiac monitors (ICMs) that include sensors capable of sensing and recording heart sounds data. ICMs are small electronic devices used for monitoring a patient's heart. ICMs can be inserted under the patient's skin and can monitor and record cardiac data for several years. Other examples of IMDs include pacemakers and cardioverter-defibrillators.

### SUMMARY

An implantable medical device (IMD) that records heart sounds data operates in a noisy environment. It can sometimes be difficult to determine whether a given dataset includes valid heart sounds data, or if the dataset includes noise. The validity of a heart sounds dataset is an important consideration before offering diagnostic information based on that heart sounds dataset. According to various aspects, the present disclosure provides techniques for determining if a heart sounds signal is valid, i.e., that it has meaningful physiological information. By using the techniques described herein, heart sounds data can be reliably verified as valid and used for any suitable purpose such as diagnosis and treatment. Heart sounds data may be used to assess systolic and diastolic function of the heart. Diagnostic metrics for tracking progression of heart failure may be derived from heart sounds. In addition, this data may be used to optimize pacing therapies to resynchronize a failing heart. That is, ventricular electrical events may be synchronized based on the heart sounds data.

In some examples, an IMD with a heart sounds sensor may be a pacemaker or other device that provides pacing therapy. A pacemaker may sense intrinsic ventricular electrical events, and deliver pacing pulses, e.g., when intrinsic ventricular events do not occur. In other examples, an IMD with a heart sounds sensor may not be configured to deliver pacing therapy, such as an insertable cardiac monitor (ICM), but is configured to sense a ventricular electrical event. In either case, a trigger, such as an instance of a ventricular electrical event, may trigger the IMD to sense (and, in some examples, store) heart sounds data. Such a recording may have any suitable duration, and each recording may include data from any suitable number of heartbeats. In one example, each recording may have a duration of about 10 seconds. The IMD may in this manner sense and record heart sounds data recordings. Such a recording is hereby referred to as an episode of heart sounds data. In the present disclosure, a heart sounds episode includes a plurality of heart sounds data segments. A heart sounds data segment is a segment or duration of time beginning with the occurrence of a ventricular electrical event and having a window of any suitable duration, e.g., in a range of 100 ms to 500 ms, and in one example, having a window of 200 ms. A window of 200 ms should be adequate to capture the S1 heart sound, which is usually one of the most dominant features of heart sounds within a cycle. Thus, a correlation between heart sounds segments may be considered largely as a correlation between S1 segments across the different cycles. In an example with a longer window, e.g., in the range of 500 ms, the window may include not only the S1 heart sound but may include at least a portion of the S2 sound as well. In this example, a correlation between heart sounds segments may be considered a correlation between not only S1 segments but part of S2 as well. In any case, when the window length is consistent across heart sounds segments, each heart sounds segment should incorporate the same phases of heart sounds across different cycles.

When a recording has been made, a processor may process the heart sounds data to determine whether the recording includes valid heart sounds data. While in some examples, the processor may be internal to the IMD, in other examples, the IMD may send the heart sounds data to an external device for processing.

For each heart sounds data segment in the recorded data, a processor may determine a correlation between the heart sounds data segment and each other heart sounds data segment in the episode. For example, if there are N heart sounds data segments in the episode, the processor may calculate correlation coefficients ρ_{x,y} representing a correlation between each heart sounds data segment and each other heart sounds data segment. Here, the first subscript x represents the index of the heart sounds data segment, where x∈{1 . . . N} ; and the second subscript y represents the other heart sounds data segments used for the correlation, where y≠x∈{1 . . . N}. For example, if there are 5 heart sounds segments in an episode, the processor calculates 4 correlation coefficients ρ for each of the 5 heart sounds segments, for a total of 20 correlation coefficients p:
ρ_{1,2}, ρ_{1,3}, ρ_{1,4}, ρ_{1,5}
ρ_{2,1}, ρ_{2,3}, ρ_{2,4}, ρ_{2,5}
ρ_{3,1}, ρ_{3,2}, ρ_{3,4}, ρ_{3,5}
ρ_{4,1}, ρ_{4,2}, ρ_{4,3}, ρ_{2,5}
ρ_{5,1}, ρ_{5,2}, ρ_{5,3}, ρ_{5,4}

Once the correlation values are calculated, the processor may then determine, for each heart sounds data segment, an average (e.g., mean, median, mode, etc.) correlation value μ with each of the other heart sounds data segments. For example, continuing with the above example with 5 heart sounds segments in an episode, the median correlation value μₓ, where x∈{1, . . . N}, is as follows:
μ₁=median (ρ_{1,2}, ρ_{1,3}, ρ_{1,4}, ρ_{1,5})
μ₂=median (ρ_{2,1}, ρ_{2,3}, ρ_{2,4}, ρ_{2,5})
μ₃=median (ρ_{3,1}, ρ_{3,2}, ρ_{3,4}, ρ_{3,5})
μ₄=median (ρ_{4,1}, ρ_{4,2}, ρ_{4,3}, ρ_{2,5})
μ₅=median (ρ_{5,1}, ρ_{5,2}, ρ_{5,3}, ρ_{5,4})

A given heart sounds data segment may be considered a valid sample if its corresponding median correlation value u is greater than a suitable correlation threshold τ, where 0<τ<1. Any suitable correlation threshold τ∈[0, 1] may be utilized within the scope of this disclosure. In some examples, the threshold may be any value from 0.7 to 0.9. By calculating the median, outliers (e.g., very high or low correlation values) may be excluded.

If at least a threshold number T (where T≤N) of heart sounds data segments are characterized as valid samples, then the recording of the heart sounds episode, including all heart sounds data segments, may be considered as valid or verified, e.g., including meaningful physiological data. Accordingly, a processor may store or transmit the recording of the heart sounds episode for use for diagnostic, treatment, or other purposes.

In some examples, a medical system is disclosed, including a heart sounds sensor, an electrical sensor/stimulus circuitry, and a processor. The heart sounds sensor is configured to sense heart sounds data. The electrical sensor circuitry is configured to sense a ventricular electrical event, or the electrical stimulus circuitry is configured to provide the ventricular electrical event. The processor is configured to record an episode of heart sounds data including a plurality of heart sounds segments, each heart sounds segment being triggered by an instance of the ventricular electrical event. For each heart sounds segment in the episode, the processor determines a correlation between the heart sounds segment and each other heart sounds segment in the episode. For each heart sounds segment in the episode, the processor then determines an average (e.g., median) correlation value with each of the other heart sounds segments in the episode. For each heart sounds segment in the episode, the processor then characterizes the heart sounds segment as a valid sample if the corresponding median correlation value is greater than a first threshold. The processor then stores the episode if at least a second threshold number of the heart sounds segments are characterized as valid samples.

This summary is intended to provide an overview of the subject matter described in this disclosure. It is not intended to provide an exclusive or exhaustive explanation of the apparatus and methods described in detail within the accompanying drawings and description below. The details of one or more aspects of the disclosure are set forth in the accompanying drawings and the description below.

### BRIEF DESCRIPTION OF DRAWINGS

FIG. 1 illustrates example environment of an example medical system in conjunction with a patient, in accordance with one or more examples of the present disclosure.

FIG. 2 is a drawing illustrating an implantable medical device (IMD) according to one or more examples of the present disclosure.

FIG. 3 illustrates an example environment of an example medical system in conjunction with a patient, in accordance with one or more examples of the present disclosure.

FIG. 4 is a block diagram illustrating internal components of an IMD according to one or more examples of the present disclosure.

FIG. 5 is a flow chart illustrating an example process for generating and storing a heart sounds episode according to one or more examples of the present disclosure.

FIG. 6 is a flow chart illustrating an example process for determining he a heart sounds episode is valid, including meaningful physiological data according to one or more examples of the present disclosure.

### DETAILED DESCRIPTION

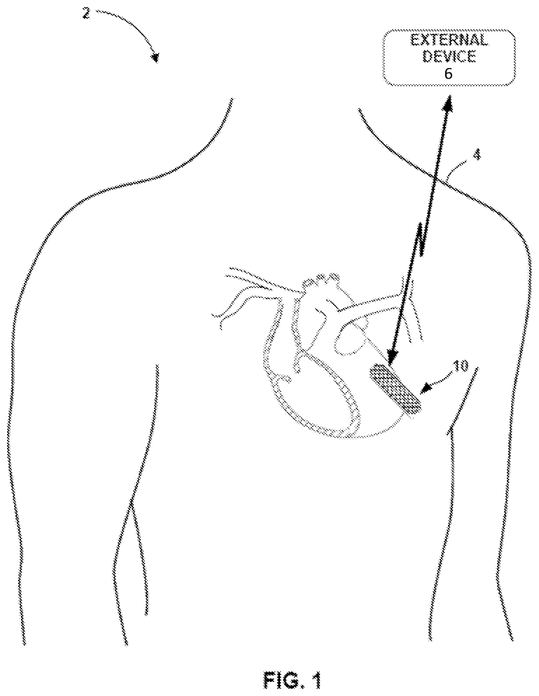

FIG. 1 illustrates the environment of an example medical system 2 in conjunction with a patient 4, in accordance with one or more techniques of this disclosure. The example techniques may be used with an implantable cardiac monitor (ICM) 10, which may be in wireless communication with at least one of external device 6 and other devices not pictured in FIG. 1. In some examples, ICM 10 is implanted outside of a thoracic cavity of patient 4 (e.g., subcutaneously in the pectoral location illustrated in FIG. 1). ICM 10 may be positioned near the sternum near or just below the level of the heart of patient 4, e.g., at least partially within the cardiac silhouette. ICM 10 may be positioned on other locations, such as patient 4's cranium region. ICM 10 includes a heart sounds sensor (not shown in FIG. 1) and is configured to sense heart sounds of the patient 4. In some examples, ICM 10 takes the form of the Reveal LINQ™ or LINQ II™ ICM. In some examples, ICM 10 includes additional sensors, such as an ECG sensor (not shown in FIG. 1) for sensing ventricular electrical activity.

External device 6 may be a computing device with a display viewable by the user and an interface for receiving user input to external device 6. In some examples, external device 6 may be a notebook computer, tablet computer, workstation, one or more servers, cellular phone, personal digital assistant, or another computing device that may run an application that enables the computing device to interact with ICM 10. External device 6 is configured to communicate with ICM 10 and, optionally, another computing device (not illustrated in FIG. 1), via wireless communication. External device 6, for example, may communicate via near-field communication technologies (e.g., inductive coupling, NFC or other communication technologies operable at ranges less than 10-20 cm) and far-field communication technologies (e.g., radiofrequency (RF) telemetry according to the 802.11 or Bluetooth® specification sets, or other communication technologies operable at ranges greater than near-field communication technologies).

External device 6 may be used to configure operational parameters and/or device settings for ICM 10. External device 6 may be used to retrieve data from ICM 10. The retrieved data may include values of physiological parameters measured by ICM 10, indications of cardiac episodes (e.g., episodes of an arrhythmia) or other maladies detected by ICM 10, and physiological signals recorded by ICM 10. In some examples, the retrieved data may include heart sounds data.

Processing circuitry of medical system 2, e.g., of ICM 10, external device 6, and/or of one or more other computing devices, may be configured to perform the example techniques for determining whether a heart sounds segment is valid, as set forth in this disclosure.

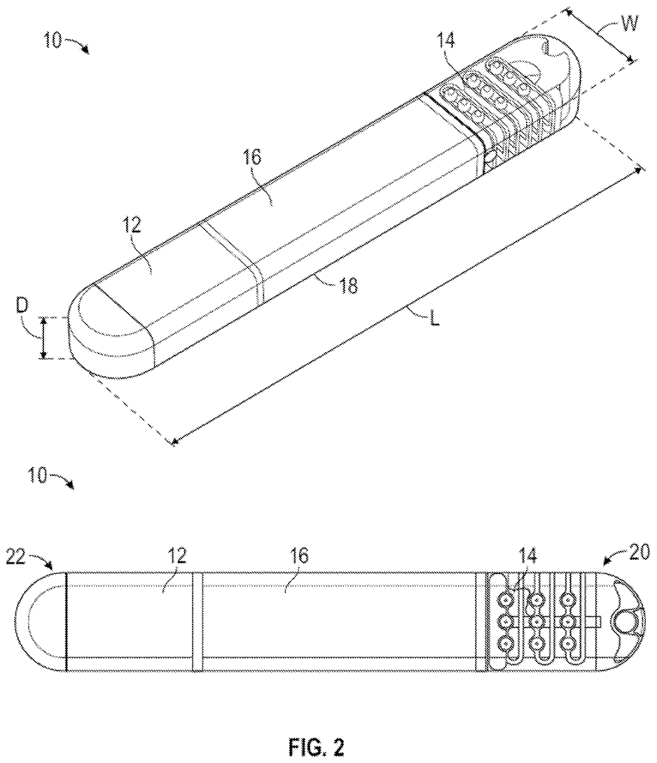

FIG. 2 is a drawing illustrating an IMD, which may be an example configuration of IMD of FIG. 1 as an ICM 10. In the example shown in FIG. 2, ICM 10 may be embodied as a monitoring device having housing 12 and a heart sounds sensor 14. Housing 12 may further include first major surface 16, second major surface 18, proximal end 20, and distal end 22. Housing 12 encloses electronic circuitry located inside the ICM 10 and protects the circuitry contained therein from body fluids. Housing 12 may be hermetically sealed and configured for subcutaneous implantation.

In the example shown in FIG. 2, ICM 10 includes a heart sounds sensor 14. Although heart sounds sensor 14 is described herein as being positioned on housing 12 of ICM 10, in other examples, heart sounds sensor 14 may be positioned on a housing of another type of IMD within patient 4, such as a transvenous, subcutaneous, or extravascular pacemaker or ICD, or connected to such a device via a lead. Further, although heart sounds sensor 14 is illustrated on first major surface 16 and a proximal end 20 of the ICM 10, heart sounds sensor 14 may be located at any suitable position such that it can detect heart sounds of the patient.

In the example shown in FIG. 2, ICM 10 is defined by a length L, a width W and thickness or depth D and is in the form of an elongated rectangular prism wherein the length L is much larger than the width W, which in turn is larger than the depth D. In one example, the geometry of the ICM 10—in particular a width W greater than the depth D—is selected to allow ICM 10 to be inserted under the skin of the patient using a minimally invasive procedure and to remain in the desired orientation during insertion. For example, the device shown in FIG. 2 includes radial asymmetries (notably, the rectangular shape) along the longitudinal axis that maintain the device in the proper orientation following insertion.

For example, ICM 10 may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 16 may range from 3 mm to 15, mm, from 3 mm to 10 mm, or from 5 mm to 15 mm, and may be any single or range of widths between 3 mm and 15 mm. The thickness or depth D of ICM 10 may range from 2 mm to 15 mm, from 2 mm to 9 mm, from 2 mm to 5 mm, from 5 mm to 15 mm, and may be any single or range of depths between 2 mm and 15 mm. In addition, ICM 10 according to an example of the present disclosure has a geometry and size designed for ease of implant and patient comfort. Examples of ICM 10 described in this disclosure may have a volume of three cubic centimeters (cm) or less, 1.5 cubic cm or less or any volume between three and 1.5 cubic centimeters.

In the example shown in FIG. 2, once inserted within the patient, the first major surface 16 faces outward, toward the skin of the patient while the second major surface 18 is located opposite the first major surface 16. In addition, in the example shown in FIG. 2, proximal end 20 and distal end 22 are rounded to reduce discomfort and irritation to surrounding tissue once inserted under the skin of the patient. ICM 10, including instrument and method for inserting ICM 10 is described, for example, in U.S. Patent Publication No. 2014/0276928, incorporated herein by reference in its entirety.

Although described in the context of examples in which an IMD that senses patient cardiac activity may comprise an ICM 10, example systems including one or more implantable, wearable, or external devices of any type configured to sense heart sounds of a patient may be configured to implement the techniques of this disclosure.

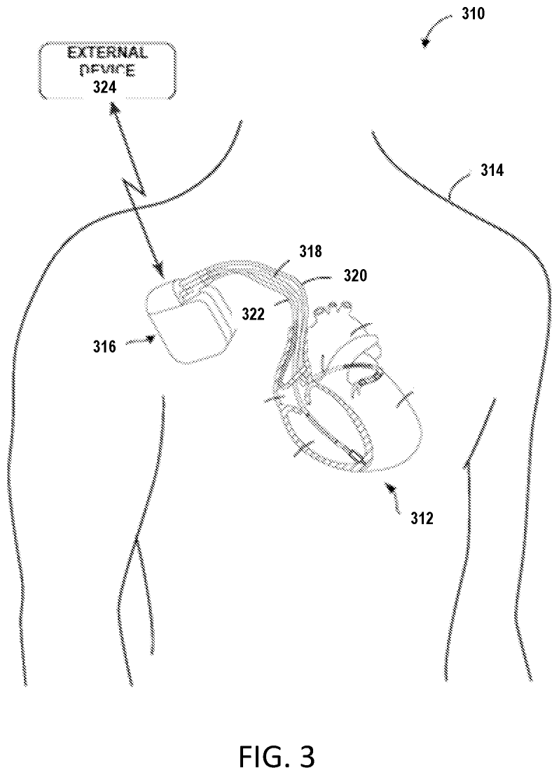

FIG. 3 illustrates the environment of an example medical system 310 in conjunction with a patient 314, in accordance with one or more techniques of this disclosure. The example techniques may be used with a pacemaker 316, which may be in wireless communication with at least one of external device 324 and other devices not pictured in FIG. 3. In the example of FIG. 3, system 310 includes a pacemaker 316, which is coupled to leads 318, 320, and 322, and an external device 324. Pacemaker 316 may be, for example, an implantable pacemaker, cardioverter, and/or defibrillator that provides electrical signals to heart 312 via electrodes coupled to one or more of leads 318, 320, and 322.

In the example of FIG. 3, leads 318, 320, 322 extend into the heart 312 of patient 314 to sense electrical activity of heart 312, e.g., one or more cardiac electrogram signals, and/or deliver electrical stimulation to heart 312. The illustrated number and positions of leads 318, 320, and 322 are examples. In other examples, pacemaker 310 may be coupled to one, two, or more than three leads that extend to a variety of positions. In some examples, system 310 may additionally or alternatively include one or more leads or lead segments (not shown in FIG. 3) that deploy one or more electrodes within the vena cava or other veins. Furthermore, in some examples, system 310 may additionally or alternatively include extravascular leads with electrodes implanted outside of heart 312, instead of or in addition to transvenous, intracardiac leads 318, 320, 322. Such leads may be used for one or more of cardiac sensing, pacing, or cardioversion/defibrillation. Additionally, in some examples, system 310 may include one or more leadless cardiac pacing devices, such as the Micra™M pacemakers commercially available from Medtronic, Inc., instead of or in addition to IMD 316.

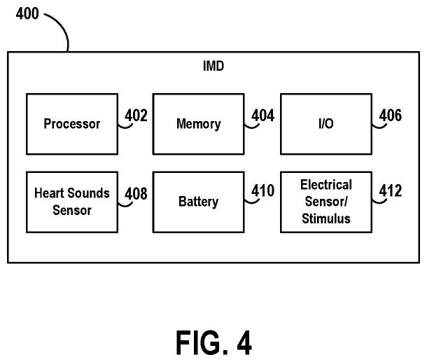

FIG. 4 is a block diagram illustrating an example of an IMD 400, which may be an example configuration of ICM 10 of FIG. 1 as an ICM and/or an example configuration of pacemaker 316 of FIG. 3. In the example shown in FIG. 4, IMD 400 includes processor 402, memory 404, input/output (transceiver) 406, heart sounds sensor 408, battery 410, and electrical sensor/stimulus circuitry 412. Processor 402 may be operatively coupled to memory 404, transceiver 406, heart sounds sensor 408, and electrical sensor/stimulus circuitry 412. The battery 410 provides operational power for processor 402, memory 404, transceiver 406, heart sounds sensor 408, and electrical sensor/stimulus circuitry 412.

Processor 402 may include fixed function circuitry and/or programmable processing circuitry. Processor 402 may include any one or more of a microprocessor, a controller, a digital signal processor (DSP), an application specific integrated circuit (ASIC), a field-programmable gate array (FPGA), or equivalent discrete or analog logic circuitry. In some examples, processor 402 may include multiple components, such as any combination of one or more microprocessors, one or more controllers, one or more DSPs, one or more ASICs, or one or more FPGAs, as well as other discrete or integrated logic circuitry. The functions attributed to processor 402 herein may be embodied as software, firmware, hardware, or any combination thereof. Processor 402 may include one or more processors that are configured to implement functionality and/or process instructions for execution within IMD 400. For example, processor 402 may be capable of processing instructions stored in memory 404.

Transceiver 406 may include any suitable hardware, firmware, software, or any combination thereof for communicating with another device, such as external device 6, another networked computing device, or another IMD or sensor. Under the control of processor 402, transceiver 406 may receive downlink telemetry from, as well as send uplink telemetry to external device 6 or another device with the aid of an internal or external antenna. In addition, processor 402 may communicate with a networked computing device via an external device (e.g., external device 6) and a computer network, such as the Medtronic CareLink® Network. Transceiver 406 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, memory 404 is a computer-readable medium that includes instructions that, when executed by processor 402, cause ICM 400 and processor 402 to perform various functions attributed to ICM 400 and processor 402 herein. Memory 404 may include any volatile, non-volatile, magnetic, optical, or electrical media, such as a random-access memory (RAM), read-only memory (ROM), non-volatile RAM (NVRAM), electrically-erasable programmable ROM (EEPROM), ferroelectric RAM (FRAM), dynamic random-access memory (DRAM), flash memory, or any other digital media.

In some examples, electrical sensor/stimulus circuitry 412 may be coupled to a set of one or more electrodes, such as electrodes 20 and 22 (FIG. 2) or electrodes 318, 320, and 322 (FIG. 3), and configured to detect ventricular electrical activity via the electrodes. Electrical sensor/stimulus circuitry 412 may include filters and amplifiers, which in some cases may be configured to detect ventricular electrical events such as R-waves and/or P-waves. Electrical sensor/stimulus circuitry 412 may include analog to digital conversion circuitry and provide a digitized version of the cardiac electrical signal to processor 402.

In some examples, electrical sensor/stimulus circuitry 412 may be configured for pacing a patient's heart via active electrical impulse signaling via the electrodes, e.g., in response to not detecting a ventricular electrical event within a programmed interval. In such examples, electrical sensor/stimulus circuitry 412 may include pulse generation circuitry, such as charge pumps, capacitors, and switches. In some examples, electrical sensor/stimulus circuitry 412 may be configured for both electrical activity sensing and for pacing.

Processor 402 may be configured to receive signals from electrical sensor/stimulus circuitry 412 and for interpreting the received signals. For example, processor 402 may be configured to detect (or receive a signal from sensor/stimulus circuitry 412 indicating detection of) a P-wave, an R-wave, or any other suitable characteristic of a sensed ventricular electrical signal. In another example, processor 402 may detect delivery of a pacing impulse by electrical sensor/stimulus circuitry 412. In still another example, processor 402 may receive any suitable signal from the electrical sensor/stimulus circuitry 412 indicating the intrinsic or paced ventricular event, such as an explicit timing signal. Processor 402 may be further configured to utilize a signal from electrical sensor/stimulus 412 (e.g., a sensed ventricular electrical signal and/or a ventricular pacing signal) as a trigger to begin recording a heart sounds segment. That is, when the processor 402 detects a suitable signal from electrical sensor/stimulus 412, the processor may be configured to use heart sounds sensor 408 to sense heart sounds data and may store the heart sounds data in memory 404.

Heart sounds sensor 408 may include any suitable sensor for acoustic sensing. Heart sounds sensor 408 may, for example, include one or more piezoelectric probes, a microphone, an accelerometer, etc., capable of acoustic sensing.

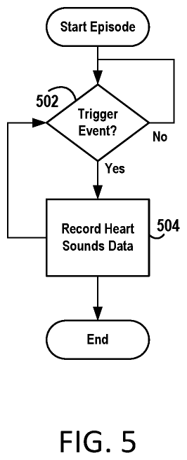

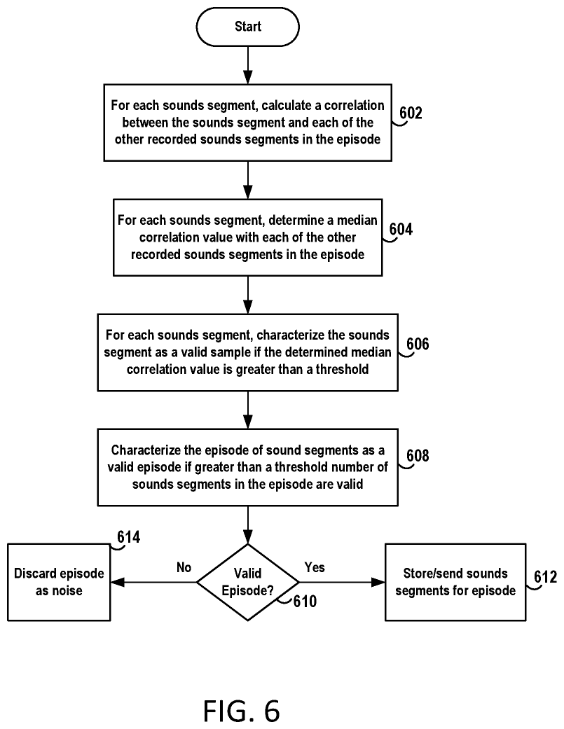

FIGS. 5 and 6 are flow charts illustrating an example process for generating and validating a heart sounds episode, including a plurality of heart sounds segments. In some examples, the process of FIGS. 5 and 6 may be carried out by an IMD, such as IMD 400 described above. In some examples, the process of FIGS. 5 and 6 may be shared between an IMD 400 and an external device 6. For example, IMD 400 may be used for sensing heart sounds, while external device 6 may be used for determining if a heart sounds episode is valid.

A heart sounds episode includes a plurality of heart sounds segments. As shown in FIG. 5, IMD 400 may start a process for recording a heart sounds episode by waiting for a trigger event (502). A trigger event may in some examples correspond to an instance of a ventricular electrical event, such as a sensed ECG signal, a pacing signal, or other suitable signal from electrical sensor/stimulus 412 to processor 402. When the IMD 400 detects a trigger event, the IMD may record heart sounds data, e.g., using heart sounds sensor 408 to sense the heart sounds data and using memory 404 to store the heart sounds data (504). In some examples, IMD 400 may store the heart sounds data along with timing of electrical pace/sense events registered by the IMD. Each recording may consist of several heart sounds segments: a segment of interest may be identified by windowing the heart sounds signal from the start of a ventricular sensing or pacing event to a fixed predetermined time period (e.g. 200 ms). For example, a heart sounds episode may include any suitable number of heart sounds segments. Further, depending on the length of the window used, each heart sounds segment may include any suitable heart sounds, such as an S1 sound, an S1 sound and an S2 sound, an S1 sound and part of an S2 sound, etc.

FIG. 6 illustrates an exemplary procedure for detecting or determining whether a heart sounds episode is valid, i.e., that it includes meaningful physiological data. As indicated above, the process of FIG. 6 may in some examples be carried out by the IMD 400, while in other examples the process of FIG. 6 may be carried out by an external processor in an external device 6. For each heart sounds segment stored in memory in the heart sounds episode, a processor may determine a correlation between the heart sounds segment and each other heart sounds segment in the episode (602). For example, the processor may calculate a correlation coefficient indicating a degree of similarity of each heart sounds segment with each other heart sounds segment in the episode. In some examples, the correlation coefficient may take a value between 0 (indicating no similarity) and 1 (indicating complete similarity). In some examples, the determined correlation coefficients may be stored in memory.

For each heart sounds segment, the processor may then determine an average (e.g., a median) correlation value between that heart sounds segment and each of the other recorded heart sounds segments in the episode (604). Thus, if there are N heart sounds segments in an episode, the processor may determine (N)(N−1) median correlation values, as described above.

For each heart sounds segment, the processor may then characterize the heart sounds segment as a valid sample if the determined median correlation value is greater than a suitable threshold (606). The threshold may be predetermined, and may depend on the number of heart sounds segments. In some examples, the threshold may be any value from 0.7 to 0.9.

If greater than a threshold number of heart sounds segments in the episode are characterized as valid, then the processor may characterize the heart sounds episode as a valid episode (608). If the heart sounds episode is valid (YES branch of 610), the processor may further analyze data from the segments within the episode to determine relevant diagnostic metrics to store for additional analysis and/or display. That is, the heart sounds data of the episode may be used for diagnosis, testing, or any other suitable purpose as the heart sounds data of the episode is characterized as representing meaningful physiological data. On the other hand, if the heart sounds episode is not valid (NO branch of 610: i.e., less than a threshold number of heart sounds segments are valid), the processor may discard the data corresponding to the heart sounds episode as noise.

Although example systems and techniques have been shown and described, it is to be understood that all the terms used herein are descriptive rather than limiting, and that many changes, modifications, and substitutions may be made by one having ordinary skill in the art without departing from the spirit and scope of the invention. The following examples are examples of systems, devices, and methods described herein.
Example 1: In some examples, a medical system includes a heart sounds sensor, at least one of an electrical sensor circuitry or an electrical stimulus circuitry, and a processor. The heart sounds sensor is configured to sense heart sounds data; the electrical sensor/stimulus is configured to sense or provide, respectively, a ventricular electrical event. The processor is configured to record an episode of heart sounds data comprising a plurality of heart sounds segments, each heart sounds segment being triggered by an instance of the ventricular electrical event. For each heart sounds segment in the episode, the processor is further configured to determine a correlation between the heart sounds segment and each other heart sounds segment in the episode. For each heart sounds segment in the episode, the processor is further configured to determine an average correlation value with each of the other heart sounds segments in the episode. For each heart sounds segment in the episode, the processor is further configured to characterize the heart sounds segment as a valid sample if the corresponding median correlation value is greater than a first threshold. The processor is further configured to store the episode if at least a second threshold number of the heart sounds segments are characterized as valid samples.
Example 2: In some examples of the medical system of Example 1, the processor, being configured to determine the average correlation value, is further configured to determine the median correlation value with each of the other heart sounds segments in the episode.
Example 3: In some examples of the medical system of Examples 1-2, the system further includes a transmitter for transmitting data corresponding to the episode to an external device.
Example 4: In some examples of the medical system of Examples 1-3, the system further includes a memory for storing data corresponding to the episode.
Example 5: In some examples of the medical system of Examples 1-4, the first threshold is between 0.7 and 0.9.
Example 6: In some examples of the medical system of Examples 1-5, the processor is further configured to assess systolic or diastolic function of a heart based on the heart sounds data.
Example 7: In some examples of the medical system of Examples 1-6, the processor is further configured to generate a diagnostic metric relating to heart failure based on the heart sounds data.
Example 8: In some examples of the medical system of Examples 1-7, the processor is further configured to synchronize the ventricular electrical event based on the heart sounds data.
Example 9: In some examples of the medical system of Examples 1-8, the system includes an implantable medical device (IMD) including the heart sounds sensor, and the at least one of the electrical sensor circuitry or the electrical stimulus circuitry.
Example 10: In some examples of the medical system of Examples 1-9, the IMD further includes the processor.
Example 11: In some examples of the medical system of Examples 1-10, the at least one of the electrical sensor circuitry or the electrical stimulus circuitry includes the electrical stimulus circuitry, and the IMD is configured to deliver cardiac pacing via the electrical stimulus circuitry.
Example 12: In some examples of the medical system of Examples 1-11, the system includes an external device including the processor.
Example 13. A method comprising: recording an episode of heart sounds data using a heart sounds sensor, the episode comprising a plurality of heart sounds segments, each heart sounds segment being triggered by an instance of a ventricular electrical event; recording ventricular electrical event data associated with the episode of heart sounds data, via at least one of a ventricular electrical sensor or a ventricular electrical stimulus; for each heart sounds segment in the episode, determining a correlation between the heart sounds segment and each other heart sounds segment in the episode; for each heart sounds segment in the episode, determining an average correlation value with each of the other heart sounds segments in the episode; for each heart sounds segment in the episode, characterizing the heart sounds segment as a valid sample if the corresponding median correlation value is greater than a first threshold; and storing the episode if at least a second threshold number of the heart sounds segments are characterized as valid samples.
Example 14. The method of Example 13, wherein determining the average correlation value comprises determining the median correlation value with each of the other heart sounds segments in the episode.
Example 15. The method of Example 13, wherein the first threshold is between 0.7 and 0.9.
Example 16. The method of Example 13, further comprising assessing systolic or diastolic function of a heart based on the heart sounds data.
Example 17. The method of Example 13, further comprising generating a diagnostic metric relating to heart failure based on the heart sounds data.
Example 18. The method of Example 13, further comprising synchronizing the ventricular electrical event based on the heart sounds data.
Example 19. A non-transitory computer-readable medium storing computer executable code comprising instructions for causing an apparatus to: record an episode of heart sounds data using a heart sounds sensor, the episode comprising a plurality of heart sounds segments, each heart sounds segment being triggered by an instance of a ventricular electrical event; record ventricular electrical event data associated with the episode of heart sounds data, via at least one of a ventricular electrical sensor or a ventricular electrical stimulus; for each heart sounds segment in the episode, determine a correlation between the heart sounds segment and each other heart sounds segment in the episode; for each heart sounds segment in the episode, determine an average correlation value with each of the other heart sounds segments in the episode; for each heart sounds segment in the episode, characterize the heart sounds segment as a valid sample if the corresponding median correlation value is greater than a first threshold; and store the episode if at least a second threshold number of the heart sounds segments are characterized as valid samples.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

1. A medical system comprising:
a heart sounds sensor configured to sense heart sounds data;
at least one of an electrical sensor circuitry configured to sense a ventricular electrical event, or an electrical stimulus circuitry configured to provide the ventricular electrical event;
a processor configured to:
record an episode of heart sounds data comprising a plurality of heart sounds segments, each heart sounds segment being triggered by a respective instance of the ventricular electrical event;
for each heart sounds segment in the episode, determine a correlation between the heart sounds segment and each other heart sounds segment in the episode;
for each heart sounds segment in the episode, determine an average correlation value with each of the other heart sounds segments in the episode;
for each heart sounds segment in the episode, characterize the heart sounds segment as a valid sample if the corresponding median correlation value is greater than a first threshold; and
store the episode if at least a second threshold number of the heart sounds segments are characterized as valid samples.

2. The system of claim 1, wherein the processor, being configured to determine the average correlation value, is further configured to determine the median correlation value with each of the other heart sounds segments in the episode.

3. The system of claim 1, wherein the first threshold is between 0.7 and 0.9.

4. The system of claim 1, wherein the processor is further configured to:
assess systolic or diastolic function of a heart based on the heart sounds data.

5. The system of claim 1, wherein the processor is further configured to:
generate a diagnostic metric relating to heart failure based on the heart sounds data.

6. The system of claim 1, wherein the processor is further configured to:
synchronize the ventricular electrical event based on the heart sounds data.

7. The system of claim 1, wherein the system comprises an implantable medical device (IMD) comprising the heart sounds sensor, and the at least one of the electrical sensor circuitry or the electrical stimulus circuitry, wherein the at least one of the electrical sensor circuitry or the electrical stimulus circuitry comprises the electrical stimulus circuitry, and wherein the IMD is configured to deliver cardiac pacing via the electrical stimulus circuitry.

8. The system of claim 7, wherein the system comprises an external device comprising the processor, wherein the IMD further comprising a transmitter for transmitting data corresponding to the episode to the external device.

9. The system of claim 1, further comprising a memory for storing data corresponding to the episode.

10. A method comprising:
recording an episode of heart sounds data using a heart sounds sensor, the episode comprising a plurality of heart sounds segments, each heart sounds segment being triggered by an instance of a ventricular electrical event;
recording ventricular electrical event data associated with the episode of heart sounds data, via at least one of a ventricular electrical sensor or a ventricular electrical stimulus;
for each heart sounds segment in the episode, determining a correlation between the heart sounds segment and each other heart sounds segment in the episode;
for each heart sounds segment in the episode, determining an average correlation value with each of the other heart sounds segments in the episode;
for each heart sounds segment in the episode, characterizing the heart sounds segment as a valid sample if the corresponding median correlation value is greater than a first threshold; and
storing the episode if at least a second threshold number of the heart sounds segments are characterized as valid samples.

11. The method of claim 10, wherein determining the average correlation value comprises determining the median correlation value with each of the other heart sounds segments in the episode.

12. The method of claim 10, wherein the first threshold is between 0.7 and 0.9.

13. The method of claim 10, further comprising assessing systolic or diastolic function of a heart based on the heart sounds data.

14. The method of claim 10, further comprising generating a diagnostic metric relating to heart failure based on the heart sounds data.

15. A non-transitory computer-readable medium storing computer executable code comprising instructions for causing an apparatus to:
record an episode of heart sounds data using a heart sounds sensor, the episode comprising a plurality of heart sounds segments, each heart sounds segment being triggered by an instance of a ventricular electrical event;
record ventricular electrical event data associated with the episode of heart sounds data, via at least one of a ventricular electrical sensor or a ventricular electrical stimulus;
for each heart sounds segment in the episode, determine a correlation between the heart sounds segment and each other heart sounds segment in the episode;
for each heart sounds segment in the episode, determine an average correlation value with each of the other heart sounds segments in the episode;
for each heart sounds segment in the episode, characterize the heart sounds segment as a valid sample if the corresponding median correlation value is greater than a first threshold; and
store the episode if at least a second threshold number of the heart sounds segments are characterized as valid samples.

16. The system of claim 7, wherein the IMD further comprises the processor.

17. The method of claim 10, further comprising synchronizing the ventricular electrical event based on the heart sounds data.
